(19) United States
(12) Patent Application Publication
(10) Pub. No.: US 2023/0112641 A1
(43) Pub. Date: Apr. 13, 2023

(54) METHODS OF TREATING DIARRHEA OR INFLAMMATORY CONDITIONS OF THE GUT

(71) Applicant: ABBOTT LABORATORIES, Abbott Park, IL (US)

(72) Inventors: Suzette PEREIRA, Westerville, OH (US); Ricardo RUEDA, Granada (ES); Vadivel GANAPATHY, Lubbock, TX (US)

(21) Appl. No.: 17/904,621

(22) PCT Filed: Mar. 11, 2021
(86) PCT No.: PCT/US2021/021864

§ 371 Date: Aug. 19, 2022

Related U.S. Application Data
(60) Provisional application No. 62/988,565, filed on Mar. 12, 2020.

(51) Int. Cl. A61K 31/19; A61K 47/42; A61K 47/26; A61K 47/44; A61P 1/12; A61P 29/00

(52) U.S. Cl. CPC A61K 31/19; A61K 47/42; A61K 47/26; A61K 47/44; A61P 1/12; A61P 29/00

(57) ABSTRACT

A method of treating diarrhea or an inflammatory condition of the gut in a subject comprises administering a therapeutically effective amount of beta-hydroxy-beta-methylbutyrate (HMB) or a salt thereof to a subject in need thereof. A method of treating secretory diarrhea in a subject comprises administering a therapeutically effective amount of HMB or a salt thereof to a subject exhibiting one or more of the following symptoms: loss of fluids from the gut, loss of electrolytes from the gut, dehydration, or inflammation of the intestinal tract.

### FIELD OF THE INVENTION

The present invention relates to methods of treating diarrhea or an inflammatory condition of the gut by administering a therapeutically effective amount of beta-hydroxy-beta-methylbutyrate (HMB) or a salt thereof to a subject in need thereof. The present invention also relates to methods of treating secretory diarrhea by administering a therapeutically effective amount of HMB or a salt thereof.

### BACKGROUND OF THE INVENTION

Diarrhea is a common condition that is characterized by frequent loose, watery stools. Diarrhea can have a number of causes, including bacterial, viral, fungal, or parasitic infections, medications, food allergies, surgery, and various digestive disorders. Side effects of diarrhea ordinarily include loose, watery stools, abdominal cramping, abdominal pain, fever, bloating, nausea, blood or mucus in the stool, loss of electrolytes, dehydration, and the urgent need to have a bowel movement. In serious cases, diarrhea can lead to malnutrition, electrolyte imbalance, and severe dehydration. In fact, electrolyte loss resulting from diarrhea is a major cause of morbidity and mortality worldwide, with the most at-risk populations being young children and the elderly. The Centers for Disease Control and Prevention indicate that roughly 2,195 children die daily of diarrhea and as many as 1 in 9 child deaths are due to diarrhea, which makes diarrhea the second leading cause of death among children that are under the age of 5.

While chronic diarrhea can last 4 weeks or more, in most cases, acute diarrhea resolves on its own within a few days. However, individuals suffering from diarrhea can ease symptoms by eating bland foods, taking over-the-counter antidiarrheal medications, and/or drinking plenty of fluids to stay hydrated. Available anti-diarrheal treatments and medications include oral rehydration solutions, probiotics, antibiotics, and/or anti-motility drugs that target intestinal motility or fluid secretion. Unfortunately, oral rehydration solutions do not normally reduce fluid loss, reduce diarrheal output, or have an effect on the duration of diarrhea. Rather, these solutions serve only to treat dehydration. Antibiotics, which are effective in reducing various symptoms of diarrhea and lessening the duration of infectious diarrheas, have a delayed onset of action and thus cannot prevent immediate dehydration. Anti-motility drugs can be used for treating noninfectious diarrhea, however they have severe side-effects in cases of infectious diarrhea. A treatment that addresses each of the above symptoms of diarrhea is therefore desirable.

In some cases, individuals may suffer from chronic diarrhea. Chronic diarrhea is a common symptom of irritable bowel disorders (IBD), the most common forms being Crohn's disease and ulcerative colitis. IBD is characterized by inflammation of the intestines and individuals suffering from IBD experience not only diarrhea, but also abdominal cramps, bloody stools, blocked bowels, fever, loss of body fluids, loss of appetite, extreme weight loss, and anemia. IBD thus has a significant impact on the daily lives of those suffering from it. Current treatment options include antibiotics, antidiarrheal drugs, lifestyle changes, and in certain situations, surgery. A nutritional intervention that can help alleviate symptoms of chronic diarrhea and treat intestinal inflammation is thus desirable.

Non-infectious cases of diarrhea may also be associated with adverse effects of drugs, particularly certain cancer treatments and HIV therapeutics. For example, chemotherapy agents tend to exacerbate gastrointestinal toxicity, which leads to diarrhea. In fact, chemotherapy induced diarrhea has been reported to affect up to 50% of colorectal cancer patients receiving 5-fluorouracil (5-FU) as single agent and severe chemotherapy induced diarrhea can develop in up to 40% of patients receiving a combination therapy. (Lee, Chun Seng, “Gastro-Intestinal Toxicity of Chemotherapeutics in Colorectal Cancer: The Role of Inflammation,” World Journal of Gastroenterology, vol. 20, no. 14, 14 April 2014, pp. 3751-3761). Diarrhea can also result from cancer itself, some examples including neuroendocrine tumors (e.g., carcinoid syndrome and Zollinger-Ellison syndrome), colon cancer, lymphoma, medullary carcinoma of the thyroid gland, and pancreatic cancer. Individuals suffering from diarrhea associated with cancer, cancer treatment, HIV therapeutics, or other drugs, are left treating diarrheal symptoms through dietary management, over-the-counter antidiarrheal medications, and/or drinking plenty of fluids to stay hydrated.

As indicated above, available antidiarrheal treatments and medications include oral rehydration solutions, probiotics, antibiotics, and/or anti-motility drugs, however each of these treatment options have drawbacks. Further, prevention methods are limited to adequate hand washing, the provision of safe water and adequate sanitation, adequate human waste disposal, and vaccination. Accordingly, improved methods of preventing dehydration resulting from diarrhea and treating diarrhea and other inflammatory conditions of the gut are desirable. A nutritional intervention that can help address the above limitations associated with existing diarrheal treatment is also desirable.

### SUMMARY OF THE INVENTION

In one embodiment, the invention is directed to a method of treating diarrhea or an inflammatory condition of the gut in a subject, comprising administering a therapeutically effective amount of beta-hydroxy-beta-methylbutyrate (HMB) or a salt thereof to a subject in need thereof.

In an additional embodiment, the present invention is directed to a method of treating secretory diarrhea in a subject, comprising administering a therapeutically effective amount of beta-hydroxy-beta-methylbutyrate (HMB) or a salt thereof to a subject exhibiting one or more of the following symptoms: loss of fluid from the gut, loss of electrolytes from the gut, dehydration, or inflammation of the intestinal tract.

The methods of treating diarrhea and inflammatory conditions of the gut according to the present invention are advantageous in that they are able to reduce the loss of fluid and/or electrolytes secreted from intestinal cells, restore electrolytes lost due to diarrhea, reduce the risk of dehydration in a subject suffering from diarrhea, prevent immediate dehydration, reduce the duration of diarrhea in a subject and/or reduce the duration of diarrhea in a subject. This is particularly advantageous in the pediatric and elderly populations, as these groups are especially vulnerable to dehydration by diarrhea. These and additional objects and advantages of the invention will be more fully apparent in view of the following detailed description.

### BRIEF DESCRIPTION OF THE DRAWINGS

The embodiments set forth in the drawings are illustrative of certain aspects of the invention and exemplary in nature and are not intended to limit the invention defined by the claims, wherein:

FIGS. 1A and B illustrate the effect of HMB on intracellular cAMP levels in colonic cells through treatment of GPR109A expressing cells with forskolin, niacin, and varying concentrations of HMB, wherein cAMP levels were measured by fluorescence, as described in Example 1.

FIGS. 2A and 2B illustrate the effect of HMB on intracellular cAMP levels in colonic cells through treatment of GPR109A expressing cells with forskolin, niacin, and varying concentrations of HMB, wherein cAMP levels were measured by radioimmunoassay, as described in Example 1.

FIG. 3 illustrates the effect of HMB on ERK phosphorylation in HMB- and niacin-treated GPR109A/NCM460D cells, as described in Example 2.

FIG. 4 illustrates the effect of HMB on formation of CD4+ FoxP3+ cells (Tregs) from a population of CD4+ T-cells using Fluorescence activated cell sorting (FACS). as described in Example 3.

FIG. 5 illustrates the effect of HMB on phosphorylation of regulatory T cells, as described in Example 3.

### DETAILED DESCRIPTION

Specific embodiments of the invention are described herein. The invention can, however, be embodied in different forms and should not be construed as limited to the embodiments set forth herein. Rather, these embodiments are provided to illustrate more specific features of certain aspects of the invention to those skilled in the art.

The terminology as set forth herein is for description of the embodiments only and should not be construed as limiting the disclosure as a whole. All references to singular characteristics or limitations of the present disclosure shall include the corresponding plural characteristic or limitation, and vice versa, unless otherwise specified or clearly implied to the contrary by the context in which the reference is made. Unless otherwise specified, “a,” “an,” “the,” and “at least one” are used interchangeably. Furthermore, as used in the description and the appended claims, the singular forms “a,” “an,” and “the” are inclusive of their plural forms, unless the context clearly indicates otherwise.

To the extent that the term “includes” or “including” is used in the description or the claims, it is intended to be inclusive of additional elements or steps, in a manner similar to the term “comprising” as that term is interpreted when employed as a transitional word in a claim. Furthermore, to the extent that the term “or” is employed (e.g., A or B), it is intended to mean “A or B or both.” When the “only A or B but not both” is intended, then the term “only A or B but not both” is employed. Thus, use of the term “or” herein is the inclusive, and not the exclusive use. When the term “and” as well as “or” are used together, as in “A and/or B” this indicates A or B as well as A and B.

The methods described in the present disclosure can comprise, consist of, or consist essentially of any of the elements and steps as described herein.

All ranges and parameters, including but not limited to percentages, parts, and ratios disclosed herein are understood to encompass any and all sub-ranges subsumed therein, and every number between the endpoints. For example, a stated range of “1 to 10” should be considered to include any and all sub-ranges beginning with a minimum value of 1 or more and ending with a maximum value of 10 or less (e.g., 1 to 6.1, or 2.3 to 9.4), and to each integer (1, 2, 3, 4, 5, 6, 7, 8, 9, and 10) contained within the range.

Any combination of method or process steps as used herein can be performed in any order, unless otherwise specified or clearly implied to the contrary by the context in which the referenced combination is made.

All percentages are percentages by weight unless otherwise indicated.

The term “dehydration” as used herein, unless otherwise specified, refers to a condition when the loss of body fluids, mostly water, exceeds the amount that is taken in. Subjects experiencing dehydration may experience symptoms including, but not limited to, dry mouth, reduced tear production, lack of sweat, muscle cramps, nausea, vomiting, heart palpitations, lightheadedness, and weakness.

The term “calcium HMB” as used herein, unless otherwise specified, refers to the calcium salt of beta-hydroxy-beta-methylbutyrate (also referred to as beta-hydroxyl-3-methyl butyric acid, beta-hydroxy isovaleric acid, or HMB), which is most typically in a monohydrate form. All weights, percentages, and concentrations as used herein to characterize calcium HMB are based on the weight of calcium HMB monohydrate, unless otherwise specified.

The term “HMB” as used herein, unless otherwise specified, refers to beta-hydroxy-beta-methylbutyrate (also referred to as beta-hydroxyl-3-methyl butyric acid, beta-hydroxy isovaleric acid) and sources thereof. All weights, percentages, and concentrations as used herein to characterize HMB are based on the weight of HMB, except that all weights, percentages, and concentrations as used herein to characterize HMB are based on the weight of HMB, unless otherwise specified.

The terms “fat” and “oil” as used herein, unless otherwise specified, are used interchangeably to refer to lipid materials derived or processed from plants or animals. These terms also include synthetic lipid materials so long as such synthetic materials are suitable for oral administration to humans.

The term “nutritional powder” as used herein, unless otherwise specified, refers to nutritional powders that are generally flowable particulates and that are reconstitutable with an aqueous liquid, and which are suitable for oral administration to a human.

The term “nutritional liquid” as used herein, unless otherwise specified, refers to nutritional products in ready-to-drink liquid form and to nutritional liquids made by reconstituting the nutritional powders described herein prior to use.

The terms “nutritional product” and “nutritional composition” as used herein, unless otherwise specified, refer to nutritional liquids and nutritional powders, the latter of which may be reconstituted to form a nutritional liquid, and are suitable for oral consumption by a human.

The term “therapeutically effective amount” as used herein, unless otherwise specified, refers to an amount of HMB that is of sufficient quantity to achieve the intended purpose of treat diarrhea or an inflammatory condition of the gut. For the purpose of the present invention, treatment of diarrhea or an inflammatory condition of the gut includes reducing the loss of fluid from the gut, reducing the loss of electrolytes from the gut, reducing diarrheal output, reducing the risk of developing dehydration, restoring lost electrolytes, reducing inflammation of the intestinal tract, eliciting tumor-suppressive effects in the colon, reducing the duration of diarrhea, or a combination thereof.

Beta-hydroxy-beta-methylbutyrate (HMB) is a naturally occurring amino acid metabolite that is known for use in a variety of nutritional products and supplements. HMB is a metabolite of the essential amino acid leucine and has been shown to modulate protein turnover and inhibit proteolysis. Calcium HMB is a commonly used form of HMB when formulated in oral nutritional products, which products include tablets, capsules, reconstitutable powders, and nutritional liquids and emulsions. Reconstitutable powders are particularly useful in this regard because such powders are often more shelf-stable than their liquid counterparts for extended periods even when formulated with multiple ingredients such as amino acids, carbohydrates, protein, and fat.

While HMB is commonly used in nutritional products to help build or maintain healthy muscle in selected individuals, the present inventors have surprisingly discovered that HMB is also useful in the treatment of diarrhea and inflammatory conditions of the gut. More particularly, the present inventors have discovered that HMB is effective in alleviating several symptoms of diarrhea and intestinal inflammation, including reducing the loss of fluid and/or electrolytes from the gut, reducing the risk of developing dehydration, restoring lost electrolytes, reducing inflammation of the intestinal tract, eliciting tumor-suppressive effects in the colon, and/or reducing the duration of diarrhea.

Depending on the cause of diarrhea, i.e., infectious or non-infectious, existing methods of treatment include replacing lost fluid and/or electrolytes via oral rehydration solutions, antibiotics, or drugs that target intestinal motility or fluid secretion, i.e., anti-motility drugs. However, as mentioned above, oral rehydration solutions fail to reduce fluid loss, diarrheal output, and the duration of diarrhea. Anti-motility drugs have severe side-effects when used to treat infectious diarrhea. Antibiotics, despite their effectiveness in reducing the symptoms and duration of diarrhea, are unable to prevent immediate dehydration due to their delayed onset of action. There is thus a need for a nutritional intervention that can help reduce the loss of fluids and electrolytes during diarrhea, particularly during secretory diarrhea, thereby lessening the risk of developing dehydration and reducing the duration of the diarrheal condition.

Hydroxycarboxylic acid receptor 2 (HCAR2), also known as niacin receptor 1 (NIACR1) or GPR109A, is a G-protein-coupled-receptor encoded by the HCAR2 gene in humans. Its activation is linked to, inter alia, the inhibition of lipolytic activity, increase in dermal blood flow, mediation of nicotinic acid-induced flushing, mediation of the antilipolytic and anti-atherogenic effects of nicotinic acid, and mediation of nicotinic acid-induced flushing. (Colletti S L et al., Hydroxycarboxylic acid receptors (version 2019.4) in the IUPHAR/BPS Guide to Pharmacology Database. IUPHAR/BPS Guide to Pharmacology CITE. 2019; 2019(4).)

GPR109A is a well-known cell-surface receptor for the B-complex vitamin niacin and is present on numerous cells, including, for example, intestinal and colonic cells, adipocytes, langerhan skin cells, kidney cells, monocytes, and macrophages. The physiological agonist for GPR109A is the ketone body β-hydroxybutyrate (β-HB) in non-colonic cells and butyrate in colonic cells. GPR109A is expressed in the lumen-facing apical membrane of the intestinal and colonic epithelial cells, and its expression level increases in the jejunum-colon axis. Maximal GPR109A expression is in the colon, where it serves as a receptor for butyrate, a bacterial metabolite generated in colonic lumen by fermentation of dietary fiber by colonic bacteria. Activation of GPR109A in the colon elicits profound anti-inflammatory effects and tumor-suppressive effects. Additionally, activation of GPR109A in intestinal cells results in a reduction of intracellular cAMP levels, which can have a significant effect on secretion of electrolytes into the lumen. (Ganapathy, V. et al., (2013) Current Opinion in Pharmacology 13: 869-874; Sivaprakasam, S. et al., (2017) Nutrients 9:E856; Singh, N. et al. Immunity 40: 128-139 (2014)). Bacterial pathogens such as Vibrio cholera and E. coli cause diarrhea by increasing the cellular levels of cAMP in intestinal and colonic epithelial cells. Therefore, agents that can reduce intracellular cAMP levels in intestinal and colonic cells will help reduce secretory diarrhea.

In addition, it has been shown that GPR109A plays an important role as a suppressor of inflammation in the colon. The mechanism associated with suppressing inflammation of the colon involves activation of GPR109A in antigen-presenting dendritic cells, which potentiates the conversion of naïve T cells into immunosuppressive regulatory T cells (Tregs) (Singh, N. et al. (2014). Immunity, 40(1), 128-139). Therefore, agents that can induce the conversion of naïve T-cells to Tregs are suitable for reducing intestinal inflammation, which is beneficial in the treatment of various inflammatory conditions of the gut, including, for example, Crohn's disease and ulcerative colitis.

In view of the above, it is desirable to have an agent that reduces intracellular cAMP levels in intestinal and colonic epithelial cells and/or induces the conversion of naïve T-cells to Tregs in order to treat diarrhea and/or intestinal inflammation of the gut.

In one embodiment, a method of treating diarrhea or an inflammatory condition of the gut in a subject is provided. The method comprises administering a therapeutically effective amount of HMB or a salt thereof to a subject in need thereof. In specific embodiments, treating diarrhea or an inflammatory condition of the gut comprises reducing the loss of fluid from the gut, reducing the loss of electrolytes from the gut, reducing diarrheal output, reducing the risk of developing dehydration, restoring lost electrolytes, reducing inflammation of the intestinal tract, eliciting tumor-suppressive effects in the colon, reducing the duration of diarrhea, or a combination thereof.

In further specific embodiments, the method is for treating an inflammatory condition of the gut selected from inflammatory bowel disease, coeliac disease, irritable bowel syndrome, acute self-limiting colitis, and colon cancer. In certain embodiments, the inflammatory condition of the gut is an inflammatory bowel disease selected from Crohn's disease and ulcerative colitis.

In another embodiment of the invention, there is provided a method of treating secretory diarrhea in subject. The method comprises administering a therapeutically effective amount of HMB or a salt thereof to a subject exhibiting one or more of the following symptoms: loss of fluid from the gut, loss of electrolytes from the gut, dehydration, or inflammation of the intestinal tract. In specific embodiments, the treatment of secretory diarrhea comprises reducing the loss of fluid from the gut, reducing the loss of electrolytes from the gut, reducing diarrheal output, reducing the risk of developing dehydration, restoring lost electrolytes, reducing inflammation of the intestinal tract, reducing the duration of secretory diarrhea in the subject, or a combination thereof.

In a further specific embodiment, the subject is a human.

Suitable sources of HMB include HMB as the free acid, a salt, including an anhydrous salt, an ester, a lactone, or other product forms that otherwise provide a bioavailable form of HMB. In further specific embodiments of the invention, the HMB or salt thereof administered to the subject is selected from the group consisting of sodium HMB, potassium HMB, magnesium HMB, chromium HMB, calcium HMB, alkali metal HMB, alkaline earth metal HMB, HMB lactone, and combinations thereof. In a particular embodiment, the HMB or salt thereof administered to the subject is provided as calcium HMB monohydrate.

In a further embodiment, the HMB or a salt thereof is administered to the subject at a daily dosage of about 0.1 to about 10 g. In a specific embodiment, HMB or a salt thereof is administered to the subject at a daily dosage of about 0.25 to 5 g. In a more specific embodiment, the HMB or salt thereof is administered to the subject at a daily dosage of about 1.5 to 3 g.

In another embodiment of the invention, the HMB or salt thereof is administered to the subject in a nutritional composition. The nutritional compositions are either formulated with the addition of HMB, most typically as a calcium monohydrate, or are otherwise prepared so as to contain HMB in the finished product. Any source of HMB is suitable for use in such compositions provided that the finished product contains HMB. In specific embodiments, such a source is calcium HMB and is most typically added as such to the nutritional products during formulation.

In a specific embodiment, the nutritional composition comprises from about 0.01 to about 10 wt % HMB or salt thereof, based on the weight of the nutritional composition. In another specific embodiment, the composition comprises from about 0.1 to about 5 wt % HMB or salt thereof, based on the weight of the nutritional composition.

The nutritional compositions may provide from about 0.1 to about 10 grams/day of HMB. Accordingly, the nutritional compositions may provide from about 0.5 to about 2.5 grams, including from about 1.0 to about 1.7 grams, including about 1.5 grams of HMB per serving, wherein a serving may be about 240 ml of ready to feed nutritional liquid or about 240 ml of reconstituted nutritional solid. In one specific embodiment, HMB is provided at a level of about 1.58 grams per 240 ml. An individual may be administered one serving per day, two servings per day, three servings per day, or four or more servings per day to receive the desired amount of HMB from the nutritional composition.

In other specific embodiments of the invention, the HMB or salt thereof is administered to the subject in a nutritional composition and the nutritional composition further comprises protein, carbohydrate, and/or fat. A wide variety of sources and types of protein, carbohydrate, and fat can be used in embodiments of nutritional compositions described herein. In a specific embodiment, the nutritional composition includes protein, carbohydrate and fat.

In further specific embodiments, the protein in the nutritional composition comprises whey protein concentrate, whey protein isolate, whey protein hydrolysate, acid casein, sodium caseinate, calcium caseinate, potassium caseinate, casein hydrolysate, milk protein concentrate, milk protein isolate, milk protein hydrolysate, nonfat dry milk, condensed skim milk, soy protein concentrate, soy protein isolate, soy protein hydrolysate, pea protein concentrate, pea protein isolate, pea protein hydrolysate, collagen protein, collagen protein isolate, rice protein, potato protein, earthworm protein, insect protein, or combinations of two or more thereof.

In other specific embodiments, the carbohydrate in the nutritional composition comprises human milk oligosaccharides (HMOs), maltodextrin, hydrolyzed starch, glucose polymers, corn syrup, corn syrup solids, rice-derived carbohydrates, sucrose, glucose, lactose, honey, sugar alcohols, isomaltulose, sucromalt, pullulan, potato starch, galactooligosaccharides, oat fiber, soy fiber, corn fiber, gum arabic, sodium carboxymethylcellulose, methylcellulose, guar gum, gellan gum, locust bean gum, konjac flour, hydroxypropyl methylcellulose, tragacanth gum, karaya gum, gum acacia, chitosan, arabinoglactins, glucomannan, xanthan gum, alginate, pectin, low methoxy pectin, high methoxy pectin, cereal beta-glucans, carrageenan, psyllium, inulin, fructooligo-saccharides, or combinations of two or more thereof. The carbohydrate can comprise digestion-resistant carbohydrates such as digestion-resistant maltodextrins, and digestion-resistant starch, slowly digestible carbohydrates.

In further specific embodiments, the fat comprises coconut oil, fractionated coconut oil, soy oil, corn oil, olive oil, safflower oil, medium chain triglyceride oil (MCT oil), high gamma linolenic (GLA) safflower oil, sunflower oil, palm oil, palm kernel oil, palm olein, canola oil, marine oils, fish oils, algal oils, borage oil, cottonseed oil, fungal oils, eicosapentaenoic acid (EPA), docosahexaenoic acid (DHA), arachidonic acid (ARA), conjugated linoleic acid (CLA), alpha-linolenic acid, interesterified oils, transesterified oils, structured lipids, and combinations of two or more thereof.

In specific embodiments of the nutritional composition, protein comprises from about 1 wt % to about 30 wt % of the nutritional composition. In more specific embodiments, the protein comprises from about 1 wt % to about 25 wt % of the nutritional composition, including about 1 wt % to about 20 wt %, about 1 wt % to about 15 wt %, about 1 wt % to about 10 wt %, about 5 wt % to about 10 wt %, or about 10 wt % to about 20 wt % of the nutritional composition. In additional specific embodiments, the protein comprises from about 1 wt % to about 5 wt % of the nutritional composition. In additional, specific embodiments, the protein comprises from about 20 wt % to about 30 wt % of the nutritional composition.

In specific embodiments of the nutritional composition, carbohydrate is present in an amount from about 5 wt % to about 75 wt % of the nutritional composition. In more specific embodiments, the carbohydrate is present in an amount from about 5 wt % to about 70 wt % of the nutritional composition, including about 5 wt % to about 65 wt %, about 5 wt % to about 50 wt %, about 5 wt % to about 40 wt %, about 5 wt % to about 30 wt %, about 5 wt % to about 25 wt %, about 10 wt % to about 65 wt %, about 20 wt % to about 65 wt %, about 30 wt % to about 65 wt %, about 40 wt % to about 65 wt %, or about 15 wt % to about 25 wt %, of the nutritional composition.

In specific embodiments, the nutritional composition comprises fat in an amount of from about 0.5 wt % to about 30 wt % of the nutritional composition. In certain specific embodiments, the fat comprises from about 1 wt % to about 30 wt % of the nutritional composition, including about 1 wt % to about 20 wt %, about 1 wt % to about 15 wt %, about 1 wt % to about 10 wt %, about 1 wt % to about 5 wt %, about 3 wt % to about 30 wt %, about 5 wt % to about 30 wt %, about 5 wt % to about 25 wt %, about 5 wt % to about 20 wt %, about 5 wt % to about 10 wt %, or about 10 wt % to about 20 wt % of the nutritional composition.

In another embodiment of the invention, the nutritional composition further comprises a nutrient selected from the group consisting of vitamins, minerals, and trace minerals. Specific embodiments of the nutritional composition may comprise vitamins and/or related nutrients, non-limiting examples of which include vitamin A, vitamin B12, vitamin C, vitamin D, vitamin E, vitamin K, thiamine, riboflavin, pyridoxine, niacin, folic acid, pantothenic acid, biotin, choline, inositol, and/or salts and derivatives thereof, and combinations thereof.

Specific embodiments of the nutritional composition comprise minerals, non-limiting examples of which include calcium, phosphorus, magnesium, zinc, manganese, sodium, potassium, molybdenum, chromium, iron, copper, and/or chloride, and combinations thereof.

According to specific embodiments, the nutritional composition is in the form of a liquid or powder and/or is administered enterally or parenterally.

The concentration of HMB in nutritional liquids may range up to about 10%, including from about 0.01% to about 10%, and also including from about 0.1% to about 5.0%, and also including from about 0.3% to about 2%, and also including from about 0.4% to about 1.5%, and also including from about 0.3% to about 0.6% by weight of the nutritional liquid. In one specific embodiment, the HMB is present in the nutritional liquid in an amount of about 0.67%, by weight of the nutritional liquid.

The total concentration of calcium HMB in nutritional powders may range up to about 10%, including from about 0.1% to about 8%, and also including from about 0.2% to about 5.0%, and also including from about 0.3% to about 3%, and also including from about 0.3% to about 1.5%, and also including from about 0.3% to about 0.6% by weight of the nutritional powder.

In specific embodiments, when the nutritional composition is a liquid, for example, reconstituted from a powder or manufactured as a ready-to-drink product, a serving ranges from about 1 ml to about 500 ml, including from about 110 ml to about 500 ml, from about 110 ml to about 417 ml, from about 120 ml to about 500 ml, from about 120 ml to about 417 ml, from about 177 ml to about 417 ml, from about 207 ml to about 296 ml, from about 230 m to about 245 ml, from about 110 ml to about 237 ml, from about 120 ml to about 245 ml, from about 110 ml to about 150 ml, and from about 120 ml to about 150 ml. In specific embodiments, the serving is about 1 ml, or about 100 ml, or about 225 ml, or about 237 ml, or about 500 ml.

In specific embodiments, when the nutritional composition is a powder, for example, a serving size is from about 40 g to about 60 g, such as 45 g, or 48.6 g, or 50 g, to be administered as a powder or to be reconstituted in from about 1 ml to about 500 ml of liquid, such as about 225 ml, or from about 230 ml to about 245 ml.

Additional specific embodiments, the nutritional composition comprises one or more components to modify the physical, chemical, aesthetic, or processing characteristics of the nutritional composition or serve as additional nutritional components. Non-limiting examples of additional components include preservatives, emulsifying agents (e.g., lecithin), buffers, sweeteners including artificial sweeteners (e.g., saccharine, aspartame, acesulfame K, sucralose), colorants, flavorants, thickening agents, stabilizers, and so forth.

The following Examples demonstrate various aspects of the invention.

### EXAMPLES

### Example 1: Effect of Hmb on Intracellular Camp Levels in Colonic Cells

This example describes the use of colonic cell lines overexpressing human GPR109A to monitor the effect of HMB on intracellular levels of cAMP as surrogate markers for activation of GPR109A. Niacin, which is the most potent agonist for the GPR109A receptor, was used as a positive control.

The activity of GI-coupled receptors like GPR109A is normally assessed by showing a reduction in cellular levels of cAMP in forskolin-treated cells. This is normally done using the commercially available kit, cAMP-Glo™ Assay. Forskolin, which is a labdane diterpenoid isolated from the Indian Coleus plant, acts on the Gs protein and activates adenyl cyclase towards increasing cellular levels of cAMP. When a GI-coupled receptor is activated in forskolin-treated cells, cAMP levels will go up.

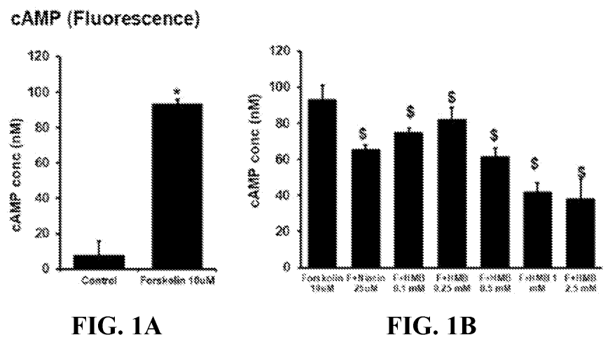

As illustrated in FIGS. 1A and 1B, treating GPR109A-expressing cells with forskolin increases cAMP levels more than 10-fold. However, when these cells were treated with niacin (25 μM) or HMB in the presence of forskolin, there was a significant reduction in the cellular levels of cAMP. As indicated above, niacin was used as a positive control for GPR109A activation. HMB at a concentration of 0.5 mM had an effect on GPR109A that is comparable to the maximal effect of niacin (EC50 for niacin is about 1 μM). The EC50 values for HMB to activate the GPR109A receptors is in submillimolar concentrations, i.e., 0.25-2.5 mM. While these values indicate low affinity, such concentrations can easily be achieved in intestinal lumen with oral dosing of HMB, as it is the luminal concentration of HMB that is relevant to activation of GPR109A present in the lumen-facing apical membrane of intestinal and colonic epithelial cells.

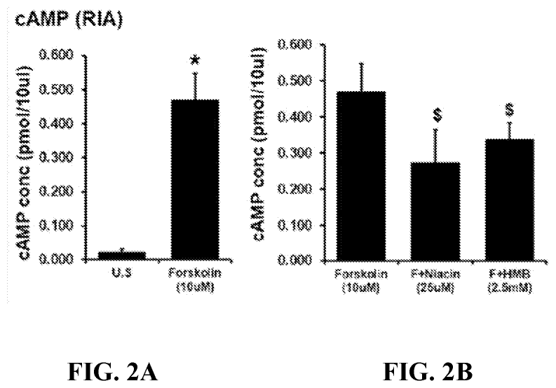

The above experiment was repeated and cAMP levels were measured by radioimmuno-assay, as illustrated in FIGS. 2A and B. Again, the control, forskolin, increased cellular levels of cAMP. Niacin and HMB decreased cellular levels of cAMP in the presence of forskolin, which confirms the effect of HMB on reducing cellular levels of cAMP in gut epithelial cells via the downregulation of adenylyl cyclase. The results thus indicate that HMB functions as an agonist for GPR109A. The ability to decrease cAMP with HMB, administered via a nutritional composition, as opposed to through use of a drug such as niacin, is advantageous.

### Example 2: Effect of Hmb on Erk Phosphorylation in Colonic Cells

This example describes the use of colonic cell lines overexpressing human GPR109A to monitor the effect of HMB on ERK phosphorylation as surrogate markers for activation of GPR109A. Niacin, which is the most potent agonist for the GPR109A receptor, was again used as a positive control. The effect of HMB on the phosphorylation of ERK as a second messenger system in the cells overexpressing GPR109A was measured.

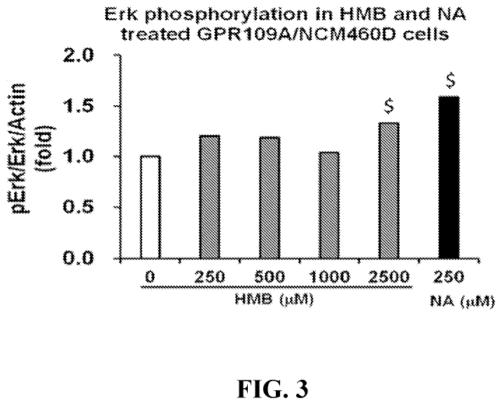

It is known that activation of GPR109A results in phosphorylation of ERK. As illustrated in FIG. 3, HMB increased phosphorylation of ERK. There was no apparent dose-response for HMB. It appears that the increase in ERK phosphorylation occurs even at an HMB concentration of 0.25 mM. Similar to Example 1 above, the results thus indicate that HMB functions as an agonist for GPR109A.

### Example 3: Effect of Hmb on Production of Regulatory T Cells

This example describes the use of immune cells (colonic dendritic cells) derived from control mice and from GPR109A-knockout mice to monitor the effect of HMB on influencing Treg formation in the small and large intestine.

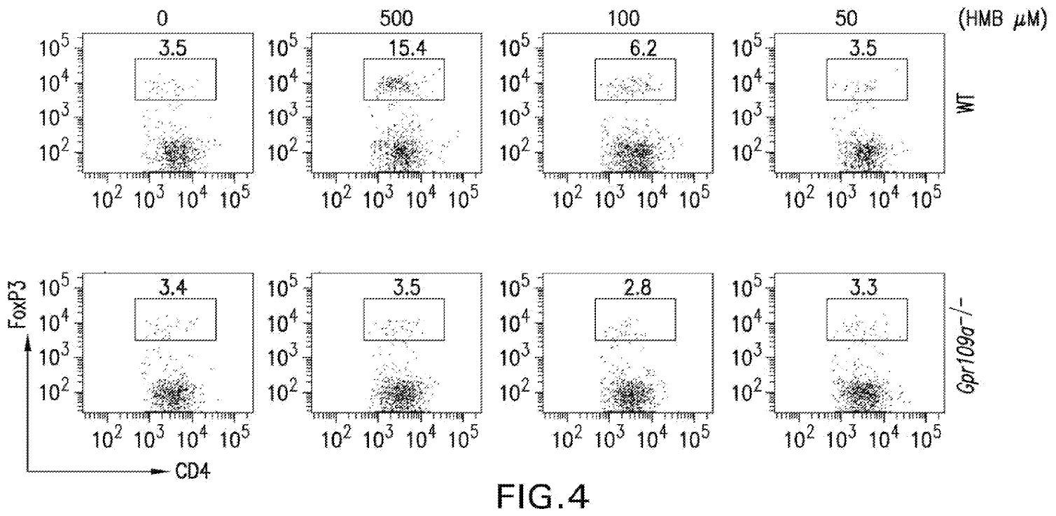

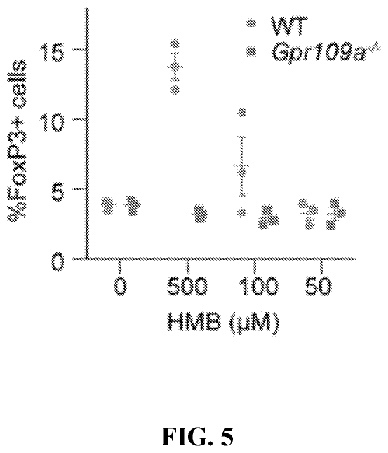

GPR109A plays an important role as a suppressor of inflammation in the colon, partly through the activation of GPR109A in antigen-presenting dendritic cells, which potentiates the conversion of naïve T cells into immunosuppressive Tregs. As indicated above, colonic dendritic cells derived from control mice and from GPR109A-knockout mice were treated with HMB. HMB at a concentration between 250-500 μM potentiated the production of Tregs in a GPR109A-dependent manner, as illustrated in FIGS. 4-5. With regard to FIG. 4, the rectangle in each FACS sorting panel identifies the CD4+ FoxP3+ cells (Tregs). The percent of these cells from the total population of CD4+ T cells is indicated on the top of the respective rectangles.

Specifically, HMB treatment at 500 μM and 100 μM showed an increase in Fox93+ Treg cells derived from wild type mice, but not GPR109A −/− knockout mice. This shows that HMB works to suppress inflammation in the intestinal tract by influencing the formation of Tregs in the intestine through the activation of GPR109A. Again, this confirms that HMB interacts with, and activates, GPR109A.

The specific embodiments and examples described herein are exemplary only and are not limiting to the invention defined by the claims.

1. A method of treating diarrhea or an inflammatory condition of the gut in a subject, comprising:
administering a therapeutically effective amount of beta-hydroxy-beta-methylbutyrate (HMB) or a salt thereof to a subject in need thereof

2. The method of claim 1, wherein the treating comprises reducing the loss of fluid from the gut, reducing the loss of electrolytes from the gut, reducing diarrheal output, reducing the risk of developing dehydration, reducing inflammation of the intestinal tract, eliciting tumor-suppressive effect in the colon, reducing the duration of diarrhea, or a combination thereof.

3. The method of claim 2, wherein the treating comprises reducing the loss of fluids from the gut, reducing the loss of electrolytes from the gut, reducing inflammation of the intestinal tract, or a combination thereof.

4. The method of claim 1, wherein the inflammatory condition of the gut is selected from the group consisting of inflammatory bowel disease, coeliac disease, irritable bowel syndrome, acute self-limiting colitis, and colon cancer.

5. The method of claim 4, wherein the inflammatory condition of the gut is an inflammatory bowel disease selected from Crohn's disease and ulcerative colitis.

6. A method of treating secretory diarrhea in a subject, comprising:
administering a therapeutically effective amount of b eta-hydroxy-beta-methylbutyrate (HMB) or a salt thereof to a subject exhibiting one or more of the following symptoms: loss of fluid from the gut, loss of electrolytes from the gut, dehydration, or inflammation of the intestinal tract.

7. The method of claim 6, wherein the treating comprises reducing the loss of fluids from the gut, reducing the loss of electrolytes from the gut, reducing diarrheal output, reducing the risk of developing dehydration, reducing inflammation of the intestinal tract, reducing the duration of secretory diarrhea in the subject, or a combination thereof.

8. The method of claim 1, wherein the HMB or salt thereof administered to the subject is selected from the group consisting of sodium HMB, potassium HMB, magnesium HMB, chromium HMB, calcium HMB, alkali metal HMB, alkaline earth metal HMB, HMB lactone and combinations thereof

9. The method of claim 8, wherein the HMB or salt thereof administered to the subject is provided as calcium HMB monohydrate.

10. The method of claim 1, wherein the HMB or a salt thereof is administered to the subject at a daily dosage of about 0.25 to 5 g.

11. The method of claim 10, wherein the HMB or salt thereof is administered to the subject at a daily dosage of about 1.5 to 3 g.

12. The method of claim 1, wherein the HMB or salt thereof is administered to the subject in a nutritional composition.

13. The method of claim 12, wherein the composition comprises from about 0.01 to about 10 wt % HMB or salt thereof, based on the weight of the nutritional composition.

14. The method of claim 13, wherein the composition comprises from about 0.1 to about 5 wt % HMB or salt thereof, based on the weight of the nutritional composition.

15. The method of claim 11, wherein the nutritional composition further comprises protein, carbohydrate, and/or a fat.

16. The method of claim 15, wherein the protein comprises whey protein concentrate, whey protein isolate, whey protein hydrolysate, acid casein, sodium caseinate, calcium caseinate, potassium caseinate, casein hydrolysate, milk protein concentrate, milk protein isolate, milk protein hydrolysate, nonfat dry milk, condensed skim milk, soy protein concentrate, soy protein isolate, soy protein hydrolysate, pea protein concentrate, pea protein isolate, pea protein hydrolysate, collagen protein, collagen protein isolate, rice protein, potato protein, earthworm protein, insect protein, or combinations of two or more thereof

17. The method of claim 15, wherein the carbohydrate comprises human milk oligosaccharides (HMOs), maltodextrin, hydrolyzed starch, glucose polymers, corn syrup, corn syrup solids, rice-derived carbohydrates, sucrose, glucose, lactose, honey, sugar alcohols, isomaltulose, sucromalt, pullulan, potato starch, galactooligosaccharides, oat fiber, soy fiber, corn fiber, gum arabic, sodium carboxymethylcellulose, methylcellulose, guar gum, gellan gum, locust bean gum, konjac flour, hydroxypropyl methylcellulose, tragacanth gum, karaya gum, gum acacia, chitosan, arabinoglactins, glucomannan, xanthan gum, alginate, pectin, low methoxy pectin, high methoxy pectin, cereal beta-glucans, carrageenan, psyllium, inulin, fructooligosaccharides, or combinations of two or more thereof

18. The method of claim 15, wherein fat comprises coconut oil, fractionated coconut oil, soy oil, corn oil, olive oil, safflower oil, medium chain triglyceride oil (MCT oil), high gamma linolenic (GLA) safflower oil, sunflower oil, palm oil, palm kernel oil, palm olein, canola oil, marine oils, fish oils, algal oils, borage oil, cottonseed oil, fungal oils, eicosapentaenoic acid (EPA), docosahexaenoic acid (DHA), arachidonic acid (ARA), conjugated linoleic acid (CLA), alpha-linolenic acid, interesterified oils, transesterified oils, structured lipids, and combinations of two or more thereof.

19. The method of claim 15, wherein the nutritional composition further comprises a nutrient selected from the group consisting of vitamins, minerals, and trace minerals.

20. The method of claim 1, wherein the nutritional composition is administered enterally or parenterally.

21. The method of claim 1, wherein the subject is a human.
